SEQUENCE LISTING (17 sequences)

SEQ ID NO: 1 - protein, 832 aa, Thermus aquaticus [sequence omitted]

SEQ ID NO: 2 - protein, 554 aa, synthetic construct
MGLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 3 - protein, 832 aa, synthetic construct
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDKNLIRVFQEGRDIHTETASWMFGVPREAVDPLMRRAAKTINFGVLYGMSAHRLSQELAIPYEEAQAFIERYFQSFPKVRAWLNKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 4 - protein, 554 aa, synthetic construct
MGLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDKNLIRVFQEGRDIHTETASWMFGVPREAVDPLMRRAAKTINFGVLYGMSAHRLSQELAIPYEEAQAFIERYFQSFPKVRAWLKKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 5 - protein, 554 aa, synthetic construct
MGLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSGDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 6 - protein, 554 aa, synthetic construct
MGLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWERLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDKNLIRVFQEGRDIHTETASWMFGVPREAVDPLMRRAAKTINFGVLYGMSAHRLSQELAIPYEEAQAFIERYFQSFPKVRAWLSKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 7 - protein, 554 aa, synthetic construct
MGLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKIRQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 8 - protein, 554 aa, synthetic construct
MGLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLIQVHDELVLEAPKERAEAVARLAKEVMEGVYPLAVPLEVEVGIGEDWLSAKE

SEQ ID NO: 9 - protein, 832 aa, synthetic construct
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSEPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 10 - protein, 832 aa, synthetic construct
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPGLIHPRTGRLHTRFNQTATATGRLSSSDPNLQNIPVRTPLGQRIRRAFVAEEGWLLVALDYSQIELRVLAHLSGDENLIRVFQEGRDIHTETASWMFGVPREAVDPQMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 11 - protein, 832 aa, synthetic construct
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLVVEVGIGEDWLSAKE

SEQ ID NO: 12 - protein, 832 aa, synthetic construct [sequence omitted]

SEQ ID NO: 13 - protein, 832 aa, synthetic construct
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPNLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 14 - protein, 554 aa, synthetic construct
MGLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDLSYLRALSLEVAEEIARLEAEVFRLAGHPFNLNSRDQLERVLFDELGLPAIGKTEKTGKRSTSAAVLEALREAHPMVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVRSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 15 - protein, 340 aa, synthetic construct
MGVPIGELLPKKELELENLNGRKVAIDAFNAIYQFLSTIRQRDGTPLMDSKGRITSHLSGLFYRTINLMEAGIKPSYVFDGKPPDFKKKELEKRAETREEALEKWEIALARGELEEAKKYAQRASKVNEILIEDAKKLLELMGIPWVQAPSEGEAQAAYMASKGDVWASASQDYDSLLFGTPKLVRNLTITGKRKLPGKDIYIEVRPELILLEDVLKELKLTREKLIELAILVGTDYNPGGIKGLGPKKALEIVKNSKDPLLKYQKTSEIDLYAIKEFFLNPPTTNDYKLEWKLPNEEDIIKFLCDEHDFSEERVKNGLERLKKATRAGKQFTLDTWFKK

SEQ ID NO: 16 - protein, 340 aa, Thermococcus kodakarensis
MGVQIGELIPRKEIELESLYGKKVAIDAFNAMYQFLSTIRQRDGTPLMDSQGRITSHLSGFFYRTINLMEAGIKPAYVFDGKPPDFKKRELEKRREAREEAEEKWYEALEKGDLEEAKKYAMRATRVNEELINDAKKLLELMGIPVVQAPSEGEAQAAYMAAKKAVYASASQDYDSLLFGAPRLVRNLTITGRRKLPGKNVYVEVKPELVVLEEVLKELGIDREKLIELAILVGTDYNPGGIKGIGPKKALTIVKRTKDPLAKYQKESDVDLYAIKEFFLNPPVTDDYELKWREPDEEGILKFLCDEHDFSEERVKNGLERLKKAVKAGKQRTLESWFGR

SEQ ID NO: 17 - DNA, 81 bp, synthetic construct
  source 1..81: mol_type = other DNA
  misc_feature 81: note = 3 prime-3 prime phosphodiester bond to thymidine
gccggccaatgtacagtattggccggcttttggcggagcgatcatctcagagcattcttagcgttttgttcttgtgtatga

(19) United States
(12) Patent Application Publication
(10) Pub. No.: US 2026/0201457 A1
(43) Pub. Date: Jul. 16, 2026

(54) PCR BY ADDITION OF A THERMOSTABLE FLAP ENDONUCLEASE

(71) Applicant: DNA POLYMERASE TECHNOLOGY, INC., St. Louis, MO (US)

(72) Inventors: Wayne M. Barnes, University City, MO (US); Katherine Rowlyk, St. Louis, MO (US)

(21) Appl. No.: 19/444,925

(22) Filed: Jan. 9, 2026

Related U.S. Application Data
(60) Provisional application No. 63/743,979, filed on Jan. 10, 2025.

(51) Int. Cl. C12Q 1/686; C12Q 1/34; C12Q 1/6823

(52) U.S. Cl. CPC C12Q 1/686; C12Q 1/34; C12Q 1/6823; G01N 2333/916

(57) ABSTRACT

Provided herein are methods for improving detection of Polymerase Chain Reaction (PCR) products comprising adding a mutant Taq DNA polymerase and a 5′-exonuclease enzyme to a PCR mixture which can amplify the polynucleotide template, wherein the mutant Taq DNA polymerase comprises a mutation that reduces or eliminates 5′-exonuclease activity of Taq. PCR mixtures of mutant Taq DNA polymerase that reduces or eliminates 5′-exonuclease activity of Taq and a 5′-exonuclease enzyme are also provided.

### CROSS REFERENCE TO RELATED APPLICATIONS

This application claims priority under 35 U.S.C. § 119 (e) to provisional patent application U.S. Ser. No. 63/743,979, filed Jan. 10, 2025. The provisional patent application is hereby incorporated by reference in its entirety herein, including without limitation: the specification, claims, and abstract, as well as any figures, tables, appendices, or drawings thereof.

### SEQUENCE LISTING INCORPORATED-BY-REFERENCE

The contents of the ST26-compliant XML file submitted electronically herewith are incorporated herein by reference in their entirety: A computer readable format copy of the Sequence Listing (filename: P15095US01.xml, date created: Jan. 9, 2026, file size 26,389 bytes).

### MATERIAL INCORPORATED BY REFERENCE

FEN1 enzymes described in U.S. Pat. No. 8,445,238 to Hall, et. al., filed on Oct. 3, 2007, and issued on May 21, 2013, the disclosure of which is incorporated herein by reference in its entirety. Additionally, FEN1 flap endonucleases are described in U.S. Pat. No. 10,487,317 to Nichols, et. al., filed on Mar. 23, 2017, and issued on Nov. 26, 2019, the disclosure of which is incorporated herein by reference in its entirety. In various embodiments, the Taq DNA polymerase can comprise mutations as described below and/or mutations disclosed in U.S. Pat. No. 10,683,537 to Kermekchiev, et. al., filed on Oct. 16, 2013 and issued on Jun. 16, 2020, the disclosure of which is incorporated herein by reference in its entirety. U.S. Pat. No. 11,814,655 is also incorporated herein by reference in its entirety.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

Taq DNA polymerase enzyme (Taq) is used to catalyze the amplification of DNA in Polymerase Chain Reactions (PCR). Mutations of Taq are known to improve its thermostability, fidelity (1), sensitivity, selectivity, speed/processivity, inhibition resistance (2), and ability to incorporate different nucleotides (3).

Known mutant Taq DNA polymerases include Omni Taq, i.e., FL-22 (SEQ ID NO: 3) (as described in U.S. Patent Application Publication No. 2011/0027832) and Omni Klentaq®, i.e., Klentaq®-10 (SEQ ID NO: 4) (as described in U.S. Patent Application Publication No. 2006/0084074).

Known mutant DNA polymerases and uses thereof are described in, for example, U.S. Pat. No. 7,462,475, issued 9 Dec. 2008; U.S. Patent Application Publication No. 2009/0170060, published 2 Jul. 2009; U.S. Patent Application Publication No. 2011/0027832, published 3 Feb. 2011; U.S. Patent Application Publication No. 2012/0028259, published 2 Feb. 2012; and international PCT application WO2012/088479, published 28 Jun. 2012, each incorporated herein by reference in their entireties.

### SUMMARY

The following objects, features, advantages, aspects, and/or embodiments are not exhaustive and do not limit the overall disclosure. No single embodiment need provide each and every object, feature, or advantage. Any of the objects, features, advantages, aspects, and/or embodiments disclosed herein can be integrated with one another, either in full or in part.

Methods for improving detection of Polymerase Chain Reaction (PCR) products, comprising: adding a mutant Thermus aquaticus (Taq) DNA polymerase to a PCR mixture comprising a polynucleotide template, primers, and deoxynucleotide triphosphates which can amplify the polynucleotide template, wherein the mutant Taq DNA polymerase comprises a mutation that reduces or eliminates 5′-exonuclease activity of Taq; and adding a 5′-exonuclease enzyme to the PCR mixture are provided.

Methods for increasing yield of PCR products in a PCR reaction, comprising: adding a mutant Taq DNA polymerase to a PCR reaction mixture comprising a polynucleotide template, primers, and deoxynucleotide triphosphates which can amplify the polynucleotide template are provided.

PCR mixtures comprising: (i) a mutant Taq DNA polymerase, wherein the mutant Taq DNA polymerase comprises a mutation that reduces or eliminates 5′-exonuclease activity of Taq; (ii) a 5′-exonuclease enzyme; and (iii) a polynucleotide template, primers which can amplify the polynucleotide template, and deoxynucleotide triphosphates are provided.

### BRIEF DESCRIPTION OF THE DRAWINGS

Those of skill in the art will understand that the drawings, described below, are for illustrative purposes only. The drawings are not intended to limit the scope of the present teachings in any way.

Several embodiments in which the present disclosure can be practiced are illustrated and described in detail, wherein like reference characters represent like components throughout the several views. The drawings are presented for exemplary purposes and may not be to scale unless otherwise indicated.

FIG. 1A is an amplification curve showing WT Taq improves TaqMan® detection by Taq D732N but not Taq D119A/D732N.

FIG. 1B is an amplification curve showing Tth DNA Polymerase improves TaqMan® detection by Taq D732N.

FIG. 2 is an amplification curve showing FEN1 allows TaqMan® detection by Taq D119A/D732N. FEN1 was included from 0 to 8 u per reaction. All amounts improved TaqMan® detection for TaqD119A/D732N

FIG. 3 is an amplification curve showing FEN1 allows TaqMan® detection by Taq D732N.

FIG. 4 is an amplification curve showing FEN1 improves TaqMan® detection by Klentaq®1.

FIG. 5A is amplification curves showing FEN1 allows TaqMan® detection by Klentaq® mutants. FEN1 was included from 0 to 8 u per reaction.

FIG. 5B is amplification curves showing FEN1 allows TaqMan® detection by Klentaq® mutants (Cesium Klentaq® AC and Cesium Klentaq® C).

FIG. 5C is amplification curves showing FEN1 allows TaqMan® detection by Klentaq® mutants (Klentaq® S and a truncated version of ZipTaq (Klentaq® version of ZipTaq)).

FIG. 6A is amplification curves showing FEN improves TaqMan® detection by Taq mutants.

FIG. 6B is amplification curves showing FEN improves TaqMan® detection by Taq mutants.

FIG. 7 is a gel showing that addition of FEN1 to PCR reactions can, in some cases, increase yield.

FIG. 8 show amplification curves showing FEN1 improves TaqMan® detection with reactions including Hot-Start Klentaq® (with aptamer) and Deep Vent® DNA polymerase (New England Biolabs, Beverly, MA, USA.

### DETAILED DESCRIPTION

In some embodiments, the terms “a” and “an” and “the” and similar references used in the context of describing a particular embodiment (especially in the context of certain of the following claims) can be construed to cover both the singular and the plural, unless specifically noted otherwise.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

As used herein, the phrases “percent sequence identity” or “percent identity” or “% sequence identity” refer to the value of the identity fraction multiplied by 100. The “identity fraction” for a sequence (e.g., a polynucleotide or polypeptide sequence) optimally aligned with a reference sequence is the number of nucleotide or amino acid residue matches in the optimal alignment, divided by the total number of nucleotides or amino acids in the full length of the entire reference sequence. An “optimal alignment” is an alignment of a sequence (e.g., a polynucleotide or polypeptide sequence) which produces the greatest number of nucleotide or amino acid residue matches across the full length of the sequence and the full length of the reference sequence.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

In certain embodiments, the present disclosure relates to compositions and methods of improving PCR assays by combining mutant Taq DNA polymerases (e.g., with mutations conferring resistance to PCR inhibitors) with a 5′-exonuclease, also called a flap endonuclease. In certain embodiments, a 5′-exonuclease, also called a flap endonuclease is used with a mutant Taq DNA polymerase to improve PCR in a probe degradation assay (e.g. a TaqMan® assay) by virtue of its ability to impart, by mixing in, a controlled amount of 5′-exonuclease to the assays where the mutant Taq DNA polymerase has 5′-exonuclease activity removed or reduced. In certain embodiments, the 5′ exonuclease can remove single stranded 5′ DNA and RNA flaps from a DNA substrates which comprises a single stranded DNA or RNA “flap” at its 5′ end and is double-stranded at its 3′ end. In various embodiments, the 5′-exonuclease can be a FEN1 enzyme (e.g., a thermostable FEN1 enzyme), including the commercially available FEN1 (New England Biolabs, Beverly, MA, USA) and/or the FEN1 enzymes described in U.S. Pat. No. 8,445,238 to Hall, et. al., filed on Oct. 3, 2007, and issued on May 21, 2013, the disclosure of which is incorporated herein by reference in its entirety. In certain embodiments, the 5′ exonuclease is a FEN1 enzyme having at least 70%, 75%, 80%, 85%, 90%, 95%, 96%, 97%, 98%, 99%, or 100% sequence identity to the polypeptide sequence of SEQ ID NO: 15 or 16 and having 5′-exonuclease (e.g., 5′ flap exonuclease) activity. In certain embodiments, the FEN1 enzyme is obtained from a Thermococcus isolate (e.g., Thermococcus 9°N™ or Thermococcus kodakarensis). Additionally, FEN1 flap endonucleases are described in U.S. Pat. No. 10,487,317 to Nichols, et. al., filed on Mar. 23, 2017, and issued on Nov. 26, 2019, the disclosure of which is incorporated herein by reference in its entirety. In various embodiments, the Taq DNA polymerase can comprise mutations as described below and/or mutations disclosed in U.S. Pat. No. 10,683,537 to Kermekchiev, et. al., filed on Oct. 16, 2013 and issued on Jun. 16, 2020, the disclosure of which is incorporated herein by reference in its entirety. The following U.S. patent applications are also incorporated herein by reference in their entireties: U.S. Pat. No. 7,462,475, issued 9 Dec. 2008; U.S. Patent Application Publication No. 2009/0170060, published 2 Jul. 2009; U.S. Patent Application Publication No. 2011/0027832, published 3 Feb. 2011; U.S. Patent Application Publication No. 2012/0028259, published 2 Feb. 2012; and international PCT application WO2012/088479, published 28 Jun. 2012.

In some embodiments a PCR mixture is formed comprising: (i) a mutant Taq DNA polymerase, wherein the mutant Taq DNA polymerase comprises one or more mutations that increases reduces or eliminates 5′-exonuclease activity of Taq; (ii) a 5′-exonuclease enzyme; and (iii) a polynucleotide template, primers which can amplify the polynucleotide template, and deoxynucleotide triphosphates. The mixture can be an assay mixture. The mixture can further comprise one or more of a sample containing a target nucleic acid (e.g., polynucleotides comprising a DNA molecule and/or an RNA molecule), primers specific for the target nucleic acid, and/or a buffer. The mixture can comprise an isolated polypeptide or a mutant DNA polymerase having inhibitor resistant DNA polymerase activity, reverse-transcriptase activity (e.g., a Taq mutant having a D732N substitution), and/or reduced or removed 5′ exonuclease activity. The mixture can amplify the target nucleic acid in the assay mixture in a PCR. In certain embodiments, the polynucleotide template is an RNA molecule, the primers include a primer for cDNA synthesis, and the PCR reaction mix further comprises a reverse transcriptase enzyme or a Taq DNA polymerase mutant with reverse-transcriptase activity (e.g., a Taq mutant having a D732N substitution). Methods for amplifying RNAs with the mutant Taq DNA polymerases and 5′ exonucleases disclosed herein can be adapted from amplification methods disclosed in U.S. Pat. No. 11,814,655, which is incorporated herein by reference in its entirety.

In some embodiments, the mutant Taq DNA Polymerase used in the PCR assay mixtures and methods provided herein has reduced or removed 5′ exonuclease activity in the presence of an inhibitory substance in an amount sufficient to cause a wild-type polymerase to fail to amplify a target nucleic acid in a polymerase chain reaction (PCR) (i.e. has inhibitor resistant DNA polymerase activity). In some embodiments, the mutant Taq DNA Polymerase has reduced or removed 5′ exonuclease activity in an amount sufficient to cause a wild type Taq polymerase of SEQ ID NO: 1 to fail to amplify a target nucleic acid in a polymerase chain reaction (PCR). In some embodiments, the inhibitory substance is contained in a sample of one or more of chocolate, peanut buffer, milk, seafood, meat, egg, plant material, blood, a blood fraction, urine, dye, soil, soil extract, humic acid, guanidinium thiocyanate (GITC), or ethanol.

In some embodiments, the mutant Taq DNA Polymerase used in the PCR assay mixtures and methods provided herein comprises an amino acid sequence having at least 95%, 96%, 97%, 98%, or 99% sequence identity to SEQ ID NO: 1 having at least one amino acid substitution selected from the group consisting of E626K, I707L, E708K, E708N, K738R, F667Y, A391T, D732N, E742R, A743R, P752S, and/or E818V (per wild-type full-length Taq numbering of SEQ ID NO: 1), or a functional fragment thereof, wherein the mutant Taq DNA Polymerase has inhibitor resistant DNA polymerase activity, reverse-transcriptase activity, and/or reduced or removed 5′ exonuclease activity. In certain embodiments the amino acid substitutions in the full length wild-type Taq DNA polymerase mutant comprise: (i) E818V; (ii) A391T, D732N, E742R, A743R, P752S, and E818V; (iii) D732N; or (iv) D119A and D732N; all per wild-type full-length Taq numbering of SEQ ID NO: 1.

In some embodiments, the mutant Taq DNA Polymerase used in the PCR assay mixtures and methods provided herein comprises an amino acid sequence at least 95%, 96%, 97%, 98%, or 99% sequence identity to SEQ ID NO: 2 having at least one amino acid substitution selected from the group consisting of E626K, I707L, E708K, E708N, K738R, F667Y, A391T, D732N, E742R, A743R, P752S, and/or E818V (per wild-type full-length Taq numbering of SEQ ID NO: 1), or a functional fragment thereof, wherein the isolated mutant Taq DNA Polymerase has inhibitor resistant DNA polymerase activity, reverse-transcriptase activity, and/or reduced or removed 5′ exonuclease activity. In certain embodiments the amino acid substitutions in the truncated Taq DNA polymerase mutant comprise: (i) K738; (ii) E626K and I707L; (iii) I707L; (iv) F667Y; or (v) A391T, D732N, E742R, A743R, P752S, and E818V, all per wild-type full-length Taq numbering of SEQ ID NO: 1.

In some embodiments, the mutant Taq DNA Polymerase used in the PCR assay mixtures and methods provided herein comprises SEQ ID NO: 3 (OmniTaq) or a polypeptide sequence having at least 95%, 96%, 97%, 98%, 99%, or 100% sequence identity to SEQ ID NO: 3 having at least one amino acid substitution selected from the group consisting of E626K, I707L, and/or E708N (per wild-type full-length Taq numbering of SEQ ID NO: 1), or a functional fragment thereof, wherein the mutant Taq DNA Polymerase has inhibitor resistant DNA polymerase activity, reverse-transcriptase activity, and/or reduced or removed 5′ exonuclease activity.

In some embodiments, the mutant Taq DNA Polymerase used in the PCR assay mixtures and methods provided herein comprises SEQ ID NO: 4 (Omni Klentaq®) or a polypeptide sequence having at least 95%, 96%, 97%, 98%, 99%, or 100% sequence identity to SEQ ID NO: 4 and having the amino acid substitutions selected from the group consisting of E626K, I707L, and/or E708K (per wild-type full-length Taq numbering of SEQ ID NO: 1), or a functional fragment thereof, wherein the mutant Taq DNA Polymerase has inhibitor resistant DNA polymerase activity, reverse-transcriptase activity, and/or reduced or removed 5′ exonuclease activity.

In some embodiments, the mutant Taq DNA Polymerase used in the PCR assay mixtures and methods provided herein comprises SEQ ID NO: 11 (mutant C-66) or a polypeptide sequence having at least 95%, 96%, 97%, 98%, 99%, or 100% sequence identity to SEQ ID NO: 11 and having the amino acid substitution E818V, or a functional fragment thereof, wherein the mutant Taq DNA Polymerase has inhibitor resistant DNA polymerase activity and reduced or removed 5′ exonuclease activity.

In some embodiments, the mutant Taq DNA Polymerase used in the PCR assay mixtures and methods provided herein comprises SEQ ID NO: 13 (mutant A-111) or a polypeptide sequence having at least 95%, 96%, 97%, 98%, 99%, or 100% sequence identity to SEQ ID NO: 13 having the amino acid substitution D732N, or a functional fragment thereof, wherein the mutant Taq DNA Polymerase has inhibitor resistant DNA polymerase activity, reverse-transcriptase activity, and/or reduced or removed 5′ exonuclease activity.

In some embodiments of the method, the assay mixture includes a dye up to about 100×, where X is a manufacturer unit for concentration for use in PCR. In some embodiments of the method, the assay mixture includes blood or a blood fraction up to about 40% of a total volume of the assay mixture. In some embodiments of the method, the assay mixture includes soil or soil extract up to about 50% of a total volume of the assay mixture or an equivalent amount that provides up to about 5 ng of humic acid per μl of the assay mixture volume. In some embodiments of the method, the assay mixture includes a bile salt, or an equivalent amount of bile, up to about 2 μg per μL of the assay mixture or up to about 20% of a total volume of the assay mixture. In some embodiments of the method, the assay mixture includes a plant material or a plant extract up to about 50% of a total volume of the assay mixture. In some embodiments of the method, the assay mixture includes urine up to about 90% of a total volume of the assay mixture. In some embodiments of the method, the assay mixture includes GITC up to about 200 mM in the assay mixture. In some embodiments of the method, the assay mixture includes ethanol up to about 10% of a total volume of the assay mixture. In some embodiments of the method, the assay mixture includes tea polyphenols up to about 12 ng per μl of assay mixture. In some embodiments of the method, the assay mixture includes tannins up to about 0.5 μg per μL of assay mixture. In some embodiments of the method, the assay mixture includes chocolate up to about 20 μg per μL of assay mixture. In some embodiments of the method, the assay mixture includes black pepper in an amount of up to 20 μg/μL of assay mixture.

In some embodiments of the method, the PCR is a real-time PCR; the assay mixture further comprises at least one dye; and amplifying the target nucleic acid comprises amplifying the target nucleic acid in the assay mixture in a real-time PCR.

In various embodiments, the methods are provided herein where a controlled amount of 5′-exonuclease is added to mutant Taq DNA polymerase enzymes which are resistant to PCR inhibitors including those present in, for example, food or food samples, such as chocolate, peanut butter, milk, seafood, meat, or egg, as well as blood, blood components, urine, humic acid, bile salts, plant tissue extracts, GITC (guanidinium) or ethanol. Such mixtures of 5′-exonucleases and Taq DNA polymerase mutants which are resistant to inhibitors and related compositions can replace key PCR components of enzyme, buffer and additives in commercially available kits, rendering them more robust and sensitive even in the presence of some PCR inhibitors, which usually can compromise detection. Also, mixtures of 5′-exonucleases and Taq DNA polymerase mutants described herein can be used directly, without requiring a commercial kit. Provided herein are compositions comprising mixtures of 5′-exonucleases and mutant Taq DNA polymerases and methods for end-point or real-time PCR analyses of samples containing inhibitory substances, such as food-containing samples, utilizing mutant polymerase enzymes that are inhibition resistant.

### Mutant Polymerases

Some embodiments of the methods and compositions provided herein comprising mixtures of 5′-exonucleases and mutant Taq DNA polymerases that can be resistant to various PCR inhibitors.

According to conventional notation, amino acid mutations discussed herein can be represented, from left to right, by the one letter code for the wild-type amino acid, the amino acid position number, and the one letter code for the mutant amino acid. For mutant polypeptide sequences, an amino acid different than corresponding wild type can be represented, from left to right, by the amino acid position number and the one letter code for the amino acid that is different than corresponding wild type.

A “variant” polypeptide described in the following paragraphs is as defined in the “variant” section further below. Exemplary sequence identity (e.g., at least about 95% sequence identity) is not meant to limit the full range of sequence identity as discussed in the “variant” section herein.

For the following discussion, wild type Taq numbering (corresponding to numbering of full-length Taq of SEQ ID NO: 1) is used in this descriptive text so as to make clear the relationship between the polypeptides. Wild type Taq (SEQ ID NO: 1) and truncated Klentaq®-1 (SEQ ID NO: 2) have complete sequence homology across positions 279-832 of SEQ ID NO: 1, except for positions 279 (Gly) and 280 (Ser) of SEQ ID NO: 1 (corresponding to positions 1 (Met) and 2 (Gly) of truncated SEQ ID NO: 2). The amino acid changes at 279-280 of wild type Taq (SEQ ID NO: 1) and positions 1-2 of truncated Klentaq®-1 (SEQ ID NO: 2) are not necessarily associated with a difference in phenotype as described herein.

With respect to wild type Taq numbering, for truncated polymerase polypeptides (e.g., Klentaq®-1 of SEQ ID NO: 2), position number 1 as notated in the Sequence Listing of SEQ ID NO: 2 corresponds to position number 279 as notated in the full-length Taq of SEQ ID NO: 1. Similarly, position number 2 of SEQ ID NO: 2 corresponds to position number 280 of SEQ ID NO: 1. Similarly, position number 554 of SEQ ID NO: 2 corresponds to position number 832 of SEQ ID NO: 1. In other words, one can determine the corresponding position in full-length SEQ ID NO: 1 by adding 278 the any position in SEQ ID NO: 2.

A mutant polymerase described herein can be produced by mutagenizing a DNA molecule which encodes the wild-type polymerase. For example, oligonucleotides providing the specific amino acid changes to a mutant polymerase described can be prepared by standard synthetic techniques (e.g., an automated DNA synthesizer) and used as PCR primers in site-directed mutagenesis. Standard procedures of expression of mutant polymerase polypeptides from encoding DNA sequences can then be performed. Alternatively, the mutant DNA polymerase polypeptides can be directly synthesized according to methods provided herein based upon the disclosure provided herein.

A mutant polymerase having a mutation described herein can be a full length mutant polymerase or a truncated mutant polymerase, as compared to a wild type Taq polymerase. For example, a truncated mutant polymerase can be truncated at position 278 per wild-type full-length Taq numbering of SEQ ID NO: 1 (e.g., position 1 of the truncated mutant corresponds to position 279 of SEQ ID NO: 1). One of skill in the art will understand that a truncated mutant polymerase can be truncated at any position of a full length sequence so long as reduced or removed 5′ exonuclease activity is retained.

A truncated mutant polymerase can be referred to as a “functional fragment” of a longer polymerase, such as a full-length polymerase. For example, SEQ ID NO: 2 (Klentaq®-1, KT-1) is a variant (having G279M and S280G per wild-type full-length Taq numbering of SEQ ID NO: 1) and functional fragment of SEQ ID NO: 1 (wild type Taq). As another example SEQ ID NO: 4 (Omni Kt, KT-10) is a functional fragment of SEQ ID NO: 3 (Omni Taq, FL-12). A functional fragment is shorter than the length of a reference polymerase and retains polymerase activity.

In some embodiments, a mutant polymerase (e.g., a full length mutant polymerase or a truncated mutant polymerase) can include one or more of the following substitutions: E626k, I707L, E708K, R487G, G418E, E708S, L533R, L781I, V453L, A454S, 1528M, K738R, F667Y, E708N, D578E, D551G, 1599V, L657Q, E404G, A391T, D732N, E742R, A743R, P752S, and E818V.

For example, a mutant polymerase can include a mutant of SEQ ID NO: 1 having one or more substitutions selected from E626k, 1707L, E708K, E708N, K738R, F667Y, A391T, D732N, E742R, A743R, P752S, and/or E818V, or a variant (e.g., at least about 95% sequence identity) thereof having at least one of these substitutions and having inhibitor resistant DNA polymerase activity, reverse-transcriptase activity, and/or reduced or removed 5′ exonuclease activity.

As another example, a mutant polymerase can include SEQ ID NO: 2 having one or more substitutions selected from E626K, I707L, E708K, E708N, K738R, F667Y, A391T, D732N, E742R, A743R, P752S, and/or E818V (per wild-type full-length Taq numbering of SEQ ID NO: 1), or a variant (e.g., at least about 95% sequence identity) thereof having at least one of these substitutions and having inhibitor resistant DNA polymerase activity, reverse-transcriptase activity, and/or reduced or removed 5′ exonuclease activity.

In some embodiments, a mutant polymerase (e.g., a full length or truncated mutant polymerase) can include an amino acid sequence of SEQ ID NO: 3 having the following substitutions: E626K, 1707L, and E708N (per wild-type full-length Taq numbering of SEQ ID NO: 1), or a variant (e.g., at least about 95% sequence identity) thereof having at least one of these substitutions and having inhibitor resistant DNA polymerase activity and reduced or removed 5′ exonuclease activity.

In some embodiments, a mutant polymerase (e.g., a full length or truncated mutant polymerase) can include an amino acid sequence of SEQ ID NO: 4 having one or more of the following substitutions: E626K, 1707L and/or E708K (per wild-type full-length Taq numbering of SEQ ID NO: 1), or a variant (e.g., at least about 95% sequence identity) thereof having at least one of these substitutions and having inhibitor resistant DNA polymerase activity and reduced or removed 5′ exonuclease activity.

In some embodiments, a mutant polymerase (e.g., a full length or truncated mutant polymerase) can include an amino acid sequence of SEQ ID NO: 5 having the substitution: R487G (per wild-type full-length Taq numbering of SEQ ID NO: 1), or a variant (e.g., at least about 95% sequence identity) thereof having the substitution and having reduced or removed 5′ exonuclease activity.

In some embodiments, a mutant polymerase (e.g., a full length or truncated mutant polymerase) can include an amino acid sequence of SEQ ID NO: 6 having one or more of the following substitutions: G418E, E626K, 1707L, and/or E708S (per wild-type full-length Taq numbering of SEQ ID NO: 1), or a variant (e.g., at least about 95% sequence identity) thereof having at least one of these substitutions and having reduced or removed 5′ exonuclease activity.

In some embodiments, a mutant polymerase (e.g., a full length or truncated mutant polymerase) can include an amino acid sequence of SEQ ID NO: 7 having the substitution: L533R (per wild-type full-length Taq numbering of SEQ ID NO: 1), or a variant (e.g., at least about 95% sequence identity) thereof having the substitution and having reduced or removed 5′ exonuclease activity.

In some embodiments, a mutant polymerase (e.g., a full length or truncated mutant polymerase) can include an amino acid sequence of SEQ ID NO: 8 having the substitution: L7811 (per wild-type full-length Taq numbering of SEQ ID NO: 1), or a variant (e.g., at least about 95% sequence identity) thereof having the substitution and having reduced or removed 5′ exonuclease activity.

In some embodiments, a mutant polymerase (e.g., a full length or truncated mutant polymerase) can include an amino acid sequence of SEQ ID NO: 9 having the substitution: D578E (per wild-type full-length Taq numbering of SEQ ID NO: 1), or a variant (e.g., at least about 95% sequence identity) thereof having the substitution and having reduced or removed 5′ exonuclease activity.

In some embodiments, a mutant polymerase (e.g., a full length or truncated mutant polymerase) can include an amino acid sequence of SEQ ID NO: 10 having one or more of the following substitutions: D551G, 1599V, and/or L657Q (per wild-type full-length Taq numbering of SEQ ID NO: 1), or a variant (e.g., at least about 95% sequence identity) thereof having at least one of these substitutions and having reduced or removed 5′ exonuclease activity.

In some embodiments, a mutant polymerase (e.g., a full length or truncated mutant polymerase) can include an amino acid sequence of SEQ ID NO: 11 having the substitution: E818V (per wild-type full-length Taq numbering of SEQ ID NO: 1), or a variant (e.g., at least about 95% sequence identity) thereof having the substitution and having inhibitor resistant DNA polymerase activity and reduced or removed 5′ exonuclease activity.

In some embodiments, a mutant polymerase (e.g., a full length or truncated mutant polymerase) can include an amino acid sequence of SEQ ID NO: 12 having the substitution: E404G (per wild-type full-length Taq numbering of SEQ ID NO: 1), or a variant (e.g., at least about 95% sequence identity) thereof having the substitution and having reduced or removed 5′ exonuclease activity.

In some embodiments, a mutant polymerase (e.g., a full length or truncated mutant polymerase) can include an amino acid sequence of SEQ ID NO: 13 having the substitution: D732N (per wild-type full-length Taq numbering of SEQ ID NO: 1), or a variant (e.g., at least about 95% sequence identity) thereof having the substitution and having inhibitor resistant DNA polymerase activity, reverse-transcriptase activity, and/or reduced or removed 5′ exonuclease activity. In certain embodiments, a mutant polymerase can comprise the N-terminally truncated DNA polymerase of SEQ ID NO: 2 having the substitution: D732N (per wild-type full-length Taq numbering of SEQ ID NO: 1), or a variant (e.g., at least about 95% sequence identity) thereof having the substitution and having inhibitor resistant DNA polymerase activity, reverse-transcriptase activity, and/or reduced or removed 5′ exonuclease activity.

In some embodiments, a mutant polymerase (e.g., a full length or truncated mutant polymerase) can include an amino acid sequence of SEQ ID NO: 14 having one or more of the following substitutions: V453L, A454S, 1528M, and/or K738R (per wild-type full-length Taq numbering of SEQ ID NO: 1), or a variant (e.g., at least about 95% sequence identity) thereof having at least one of these substitutions and having reduced or removed 5′ exonuclease activity.

A mutant polymerase described herein can be used in conjunction with compositions or processes described in U.S. Pat. Nos. 6,403,341; 7,393,635; 7,462,475; WO 2012/088479 (and corresponding U.S. application Ser. No. 13/997,194); and US Pat App Pub No. 2012/0028259, each incorporated herein by reference.

### Variants

The term “variant” polypeptides (or encoding polynucleotides) is discussed below. The description of “variant” below is incorporated by reference into each recitation of “variant” in the description of mutant polymerases herein. For example, the full range of sequence identity discussed below applies equally to “variant” polypeptides discussed elsewhere herein.

Included in the scope of the present disclosure are variant polypeptides (or encoding polynucleotides) with at least 80% sequence identity to sequences described herein, so long as such variants retain a polymerase activity (e.g., a resistant polymerase activity).

For example, a variant polypeptide (or an encoding polynucleotide) with polymerase activity can have at least about 85%, 86%, 87%, 88%, 89%, 90%, 91%, 92%, 93%, 94%, 95%, 96%, 97%, 98%, 99%, or 99.9% sequence identity to sequences disclosed herein (including disclosed sequences having substitutions described herein). It is understood that in some embodiments, “about” modifies each of these recited sequence identity values. A variant polypeptide (or encoding polynucleotides) with polymerase activity can have at least 95% sequence identity to a sequence disclosed herein. A variant polypeptide (or an encoding polynucleotide) with reduced or removed 5′ exonuclease activity can have at least 95%, 96%, 97%, 98%, or 99% sequence identity to a sequence disclosed herein. In certain embodiments, variant species are representative of the genus of variant polypeptides of each of these respective sequences because the variants possess the specified catalytic activity (e.g., inhibitor resistant DNA polymerase activity, reverse-transcriptase activity, and/or reduced or removed 5′ exonuclease activity) and have the percent identity required above to the reference sequence.

Design, generation, and testing of the variant polypeptides having the above required percent identities to the sequences of the mutant DNA polymerases and retaining a required resistant phenotype is within the skill of the art. For example, directed evolution and rapid isolation of mutants can be according to methods described in references including, but not limited to, Link et al. (2007) Nature Reviews 5 (9), 680-688; Sanger et al. (1991) Gene 97 (1), 119-123; Ghadessy et al. (2001) Proc Natl Acad Sci USA 98 (8) 4552-4557.

Thus, one skilled in the art could generate a large number of polypeptide variants having, for example, at least 95-99% identity to the sequences of mutant DNA polymerases described herein and screen such for phenotypes including, dye-resistance, blood-resistance, or soil-resistance according to methods routine in the art. Generally, conservative substitutions can be made at any position so long as the required activity is retained.

TABLE 1
Sequence Listings of and Descriptions of
Wild-Type and Mutant Enzymes, Aptamer.

| | AA | |
|---|---|---|
| SEQ ID NO | Changes¹ | Comments |
| SEQ ID NO: 1 | None | Full-length WT Taq DNA polymerase |
| SEQ ID NO: 2 | None | KlenTaq ® Fragment of DNA polymerase |
| SEQ ID NO: 3 | E626K | OmniTaq |
| | I707L | |
| | E708N | |
| SEQ ID NO: 4 | E626K | Omni Klentaq ® |
| | I707L | |
| | E708K | |
| SEQ ID NO: 5 | R487G | Taq DNA polymerase Mutant B-9 |
| SEQ ID NO: 6 | G418E | Taq DNA polymerase Mutant H-10 |
| | E626K | |
| | 1707L | |
| | E708S | |
| SEQ ID NO: 7 | L533R | Taq DNA polymerase Mutant F-12 |
| SEQ ID NO: 8 | L7811 | Taq DNA polymerase Mutant E-12 |
| SEQ ID NO: 9 | D578E | Taq DNA polymerase Mutant C-6 |
| SEQ ID NO: 10 | D551G | Taq DNA polymerase Mutant C-12 |
| | I599V | |
| | L657Q | |
| SEQ ID NO: 11 | E818V | Taq DNA polymerase Mutant C-66 |
| SEQ ID NO: 12 | E404G | Taq DNA polymerase Mutant H-2 |
| SEQ ID NO: 13 | D732N | OmniTaq 2 |
| SEQ ID NO: 14 | V453L | Taq DNA polymerase Mutant H-101 |
| | A454S | |
| | I528M | |
| | K738R | |
| SEQ ID NO: 15 | Not | Thermococcus FEN1 (multispecies |
| | applicable | consensus; NCBI Reference |
| | | Sequence: WP_283217706.1) |
| SEQ ID NO: 16 | None | Thermococcus kodakarensis FEN1 |
| SEQ ID NO: 17 | | DNA hot-start aptamer |

¹AA changes are according to the full-length Wild- type Taq DNA Polymerase numbering.

### Dyes

Various embodiments of the mutant polymerase enzymes described herein can tolerate increased concentrations of dyes, such as those used in real-time PCR (qPCR). A mutant polymerase can be used to amplify a DNA target in a real-time PCR of a DNA target in the presence of an inhibitory dye. A mutant polymerase can be used in combination with an enzyme having reverse transcriptase activity to amplify an RNA target in a real-time reverse transcriptase (RT) PCR of an RNA target in the presence of an inhibitory dye. Such increased concentrations include, but are not limited to, up to about 0.5×, 1×, 1.5×, 2×, 2.5×, 3×, 3.5×, 4×, 4.5×, 5×, 5.5×, 6×, 6.5×, 7×, 7.5×, 8×, 8.5×, 9×, 9.5×, 10×, 15×, 20×, 25×, 30×, 35×, 40×, 45×, 50×, 55×, 60×, 65×, 70×, 80×, 90×, or 100×, or even higher over the dye concentration conventionally used in the assay. As an example, X can be the standard manufacturers unit for dye concentration provided in a commercial product (e.g., SYBR Green, Molecular Probes, Eugene, Oregon). For example, for SYBR Green, X corresponds to a concentration of about 10 μM.

Dye-resistance can be readily determined by assays which can be adapted from assays described in US Pat App Pub No. 2011/0027832, which is incorporated herein by reference in its entirety.

Dyes for use in the methods described herein include, but are not limited to, SYBR Green (Molecular Probes, Eugene, Oregon), LC Green (Idaho Technology, Salt Lake City, Utah), Pico Green (Molecular Probes, Eugene, Oregon), TOTO (Molecular Probes, Eugene, Oregon), YOYO (Molecular Probes, Eugene, Oregon) and SYTO9 (Molecular Probes, Eugene, Oregon).

A dye can be a nucleic acid intercalating dye. A nucleic acid intercalating dye is understood to be a molecule that bind to nucleic acids in a reversible, non-covalent fashion, by insertion between the base pairs of the double helix, thereby indicating the presence and amount of nucleic acids.

Generally, nucleic acid intercalating dyes are planar, aromatic, ring-shaped chromophore molecules. In some embodiments, intercalating dyes include fluorescent dyes. Numerous intercalating dyes can be adapted for use in the methods provided herein. Some non-limiting examples include PICO GREEN (P-7581, Molecular Probes), EB (E-8751, Sigma), propidium iodide (P-4170, Sigma), Acridine orange (A-6014, Sigma), 7-aminoactinomycin D (A-1310, Molecular Probes), cyanine dyes (e.g., TOTO, YOYO, BOBO, and POPO), SYTO, SYBR Green I, SYBR Green II, SYBR DX, Oli Green, CyQuant GR, SYTOX Green, SYT09, SYTOIO, SYTO 17, SYBRI 4, FUN-I, DEAD Red, Hexidium Iodide, Dihydroethidium, Ethidium Homodimer, 9-Amino-6-Chloro-2-Methoxyacridine, DAPI, DIPI, Indole dye, Imidazole dye, Actinomycin D, Hydroxystilbamidine, LOS 751 (U.S. Pat. No. 6,210,885), BOXTO, LC Green, Evagreen, and Bebo.

With their tolerance to high dye concentrations, the mutant polymerases described herein can outperform other conventional polymerase enzymes, including top commercial PCR enzymes, with commercially available dyes used in qPCR including, but not limited to, SYBR Green, LC Green (Idaho Technology, Salt Lake City, Utah), PICO, TOTO (Molecular Probes, Eugene, Oregon), YOYO (Molecular Probes, Eugene, Oregon), SYTO (Molecular Probes, Eugene, Oregon), and ethidium bromide. Some of these dyes are even more inhibitory than SYBR Green to a conventional Taq enzyme in PCR.

### Blood

In some embodiments, a mutant polymerase described herein can amplify a target nucleic acid in the presence of blood or blood components. A mutant polymerase can be used to amplify a DNA target in a real-time PCR of a DNA target in the presence of blood or blood components. A mutant polymerase can be used in combination with an enzyme having reverse transcriptase activity to amplify an RNA target in a real-time reverse transcriptase (RT) PCR of an RNA target in the presence of blood or blood components.

Blood-resistance can be readily determined by assays described in US Pat App Pub No. 2006/0084074 or US Pat App Pub No. 2011/0027832.

Whole blood generally comprises plasma, serum, and blood cells. Blood components include, but are not limited to, red blood cells, white blood cells (e.g., leukocytes or platelets, i.e., thrombocytes), plasma, serum, hemoglobin, water, proteins, glucose, amino acids, fatty acids, mineral ions, hormones, carbon dioxide, urea, and lactic acid. A mutant polymerase described herein can be used in PCR to amplify a nucleic acid target in the presence of one or more such blood components.

Blood plasma is generally understood as a liquid suspension in which blood cells are circulated. Thus, blood plasma can include one or more of water, proteins, glucose, amino acids, fatty acids, mineral ions, hormones, carbon dioxide, urea, lactic acid, platelets (i.e., thrombocytes), and blood cells. In a human subject, blood plasma represents about 55% of whole blood, or about 2.7 to 3 liters in an average human subject. Blood plasma contains about 92% water, 8% blood plasma proteins, and trace amounts of other materials. Blood plasma can contain serum albumin, blood-clotting factors, immunoglobulins, lipoproteins, other proteins, and electrolytes (e.g., sodium and chloride). A crude sample comprising blood plasma can also contain blood cells. A mutant polymerase described herein can be used in PCR to amplify a nucleic acid target in the presence of blood plasma.

Blood serum is generally understood as plasma from which clotting proteins have been removed, leaving mostly albumin and immunoglobulins. A mutant polymerase described herein can be used in PCR to amplify a nucleic acid target in the presence of blood serum.

In some embodiments, a mutant polymerase can display amplification activity in PCR assays (e.g., end point or real-time PCR) containing from about 1% to about 40% whole blood in the reaction mixture (vol/vol). For example, whole blood can comprise at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, or 40% of a total volume of a PCR assay mixture comprising a mutant polymerase described herein. In contrast, the full-length wild-type Taq enzyme (SEQ ID NO: 1) is usually completely inhibited in a blood concentration range of about 0.004% to about 0.2% whole blood in the reaction mixture (vol/vol).

In some embodiments, a mutant polymerase can display amplification activity in PCR assays (e.g., end point or real-time PCR) containing from about 1% to about 25% blood plasma in the reaction mixture (vol/vol). For example, blood plasma can comprise at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% of a total volume of a PCR assay mixture comprising a mutant polymerase described herein.

In some embodiments, a mutant polymerase can display amplification activity in PCR assays (e.g., end point or real-time PCR) containing from about 1% to about 25% blood serum in the reaction mixture (vol/vol). For example, blood serum can comprise at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% of a total volume of a PCR assay mixture comprising a mutant polymerase described herein.

### PCR

A mutant polymerase (including all variants thereof) described herein can be used in a variety of polymerase reactions. Polymerase chain reaction conditions which can be adapted for use in the methods include those described in Dorak (2006) Real-Time PCR, Taylor & Francis, ISBN 041537734X; Bustin, ed. (2004) A-Z of Quantitative PCR, International University Line, ISBN 0963681788; King and O'Connel (2002) RT-PCR Protocols, 1^{st} Ed., Human Press, ISBN-10 0896038750). For example, a mutant polymerase and a 5′ exonuclease can be employed in PCR reactions, primer extension reactions, etc.

For example, a mutant polymerases and a 5′ exonuclease described herein can be used in nucleic acid amplification processes (either alone or in combination with one or more other enzymes), such as Allele-specific PCR; Assembly PCR or Polymerase Cycling Assembly; Asymmetric PCR; Linear-After-The-Exponential-PCR; Helicase-dependent amplification; Hot-start PCR; intersequence-specific PCR; Inverse PCR; Ligation-mediated PCR; Methylation-specific PCR; Miniprimer PCR; Multiplex Ligation-dependent Probe Amplification; Multiplex-PCR; Nested PCR; Overlap-extension PCR; Quantitative PCR; Quantitative End-Point PCR; Quantitative Real-Time PCR; RT-PCR (Reverse Transcription PCR); Solid Phase PCR; Thermal asymmetric interlaced PCR; Touchdown PCR; PAN-AC; Universal Fast Walking; Long PCR; Rapid Amplified Polymorphic DNA Analysis; Rapid Amplification of cDNA Ends (RACE); Differential Display PCR; In situ PCR; High-Fidelity PCR; PCR or DNA Sequencing (cycle sequencing).

A target nucleic acid of a sample can be any target nucleic acid of interest. For example, a target nucleic acid can be a deoxyribonucleic acid (DNA), a ribonucleic acid (RNA), or an artificial nucleic acid analog (e.g., a peptide nucleic acid, morpholino- and locked nucleic acid, glycol nucleic acid, or threose nucleic acid).

A primer is understood to refer to an oligonucleotide, whether occurring naturally or produced synthetically, which is capable of acting as a point of initiation of nucleic acid synthesis when placed under conditions in which synthesis of a primer extension product which is complementary to a nucleic acid strand is induced, e.g., in the presence of four different nucleotide triphosphates and thermostable enzyme in an appropriate buffer (“buffer” includes pH, ionic strength, cofactors, etc.) and at a suitable temperature. The primer is preferably single-stranded for maximum efficiency in amplification, but can alternatively be double-stranded. If double-stranded, the primer is first treated to separate its strands before being used to prepare extension products. Preferably, the primer is an oligodeoxyribonucleotide. The primer must be sufficiently long to prime the synthesis of extension products in the presence of the thermostable enzyme.

The exact lengths of the primers will depend on many factors, including temperature, source of primer and use of the method. For example, depending on the complexity of the target sequence, the oligonucleotide primer typically contains 15-25 nucleotides, although it can contain more or few nucleotides. Short primer molecules generally require colder temperatures to form sufficiently stable hybrid complexes with template.

A target nucleic acid, e.g., a template DNA molecule, is understood to be a strand of a nucleic acid from which a complementary nucleic acid strand can be synthesized by a DNA polymerase, for example, in a primer extension reaction.

In some embodiments, the use of a mutant polymerase enzyme described herein does not require any, or substantial, changes in the typical protocol, but can allow, for example, for the presence of higher concentrations of inhibitory substances. A mutant polymerase described herein, and methods for use thereof, can allow for elimination or substantial elimination of an enrichment step for sample preparation. Eliminating an enrichment step can significantly reduce the time to detection or quantification.

A mutant polymerase and a 5′ exonuclease described herein can be used in an end-point PCR. For example, end-point PCR is commonly carried out in a reaction volume of about 10-200 μl in small reaction tubes (about 0.2-0.5 ml volumes) in a thermal cycler.

A mutant polymerase and a 5′ exonuclease described herein can be used with a variety of commercially available end-point PCR kits. The use of a mutant polymerase enzyme described herein generally does not require any, or substantial, changes in the typical end-point PCR protocol, but can allow, for example, a sample having a higher amount of an inhibitory substance.

A mutant polymerase and a 5′ exonuclease described herein can be used in real-time PCR (also known as a quantitative polymerase chain reaction (qPCR)). For example, a mutant polymerase described herein can be used in a real-time PCR assay featuring a non-specific fluorescent dye (e.g., a fluorochrome) that can intercalate with any double-stranded DNA. With a non-specific fluorescent dye, an increase in DNA product during PCR can lead to an increase in fluorescence intensity and is measured at each cycle, thus allowing DNA concentrations to be quantified.

As another example, a mutant polymerase and a 5′ exonuclease described herein can be used in a real-time PCR assay featuring a hybridization probe. As another example, a mutant polymerase described herein can be used in a real-time PCR multiplex assay featuring a hybridization probe. A hybridization probe can be a sequence-specific DNA probe including a fluorescent reporter at one end and a quencher of fluorescence at the opposite end of the probe, where break down of the probe by a 5′ to 3′ exonuclease activity of a polymerase can break the reporter-quencher proximity and thus allow unquenched emission of fluorescence, which can be detected after excitation with a laser (e.g., a TaqMan® assay). With a hybridization probe, an increase in the product targeted by the reporter probe at each PCR cycle can cause a proportional increase in fluorescence due to the breakdown of the probe and release of the reporter. A mutant polymerase and a 5′ exonuclease described herein can be used with a variety of commercially available real-time PCR kits.

Thus, methods described herein can be applied to improve the nucleic acid detection in an end-point PCR or a real-time PCR.

In some embodiments, a mutant polymerase and a 5′ exonuclease described herein can be used in combination with an enzyme having reverse transcriptase activity in a real-time reverse transcriptase (RT) PCR amplification of an RNA target. It is noted that reverse transcriptase (RT) PCR is not to be confused with real-time polymerase chain reaction (Q-PCR), which is sometimes (incorrectly) abbreviated as RT-PCR in the art. In RT-PCR, an RNA strand is first reverse transcribed into its DNA complement (complementary DNA, or cDNA) using the enzyme reverse transcriptase, and the resulting cDNA is amplified using traditional PCR. Like with end-point PCR, conventional RT-PCR protocols require extensive purification steps prior to amplification to purify RNA from inhibitors and ribonucleases, which can destroy the RNA template. Both the inhibition and degradation of RNA are major concerns in important clinical and diagnostics tests, which can lead to false-negative results.

The buffer for use in the various PCR assay mixtures described herein is generally a physiologically compatible buffer that is compatible with the function of enzyme activities and enables cells or biological macromolecules to retain their normal physiological and biochemical functions. Typically, a physiologically compatible buffer will include a buffering agent (e.g., TRIS, MES, PO4, HEPES, etc.), a chelating agent (e.g., EDTA, EGTA, or the like), a salt (e.g., ammonium sulfate, NaCl, KCl, MgCl₂, CaCl₂), NaOAc, KOAc, Mg(OAc)₂, etc.) and optionally a stabilizing agent (e.g., sucrose, glycerine, Tween20, etc.).

Various PCR additives and enhancers can be employed with the methods described herein. For example, betaine (e.g., MasterAmp™ 10×PCR, Epicentre Biotechnologies) can be added to the PCR assay, to further aid in overcoming the inhibition by inhibitory substances described herein. Betaine can be included at final concentration from about 1M to about 2M. Generally, betaine alone is insufficient to overcome the inhibition of various inhibitory substances described herein when used with conventional DNA polymerases.

As another example, a mutant polymerase and a 5′ exonuclease described herein can be used in conjunction with a PCR enhancer described in US Pat Pub No. 2012/0028259 or WO 2012/088479, each incorporated herein by reference. For example, a mutant polymerase can be used in conjunction with a PCR enhancer including trehalose (e.g., about 0.1 to about 1.0 M D-(+)-trehalose per amplification reaction mixture volume), carnitine (about 0.1 to about 1.5 M L-carnitine per amplification reaction mixture volume), or a non-ionic detergent (e.g., Brij-58, NP-40, Nonidet P-40, lgepal CA-630, Brij-58, Tween-20, NP-40, or Triton X-100 at about 0.01% to about 8% non-ionic detergent per amplification reaction mixture volume) or optionally one or more of heparin (e.g., an amount of heparin equivalent to about 2 units to about 50 units heparin per ml of whole blood, plasma, or serum in an amplification reaction mixture), casein (at least about 0.05% up to about 2.5% per amplification reaction mixture volume), or polyvinylpyrrolidone (PVP) or a modified polymer of PVP (PVPP) (e.g., about 0.1% up to about 25%). As another example, a mutant polymerase can be used in conjunction with a PCR enhancer including about 0.6 M trehalose per amplification reaction mixture volume; about 0.5 M carnitine per amplification reaction mixture volume; or a non-ionic detergent (e.g., a polyoxyethylene cetyl ether at about 0.04% to about 0.2% or a nonyl phenoxylpolyethoxylethanol at about 0.4% to about 0.8% per amplification reaction mixture volume); or optional heparin at about 10 units per ml of whole blood, blood fraction, plasma, or serum.

As another example, a mutant polymerase and a 5′ exonuclease described herein can be used in conjunction with commercially available PCR amplification reaction enhancers, such as MasterAmp™ 10×PCR Enhancer, Epicentre Biotechnologies; TaqMaster PCR Enhancer, MasterTaq Kit, PCR Extender System, 5 PRIME GmbH; Hi-Spec Additive, Bioline; PCRboost®, Biomatrica®; PCRX Enhancer System, Invitrogen; Taq Extender™ PCR Additive, Perfect Match PCR Enhancer, Stratagene; Polymer-Aide PCR Enhancer, Sigma-Aldrich.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to a mutant DNA polymerase and a 5′ exonuclease described herein or a nucleic acid encoding such mutant polymerase and 5′ exonuclease or, optionally, a primer, a buffer, or other composition or component (e.g., a magnesium salt) necessary or helpful for PCR. In certain embodiments, the mutant DNA polymerase and 5′ exonuclease are provided in separate containers. In certain embodiments, the mutant DNA polymerase and 5′ exonuclease are provided in single containers (e.g., in a concentrated form suitable for combining with other PCR mixture components to achieve a suitable final concentration in the PCR). Such packaging of the components separately can, if desired, be presented in a pack or dispenser device which can contain one or more assay unit forms containing a composition. The pack can, for example, comprise metal or plastic foil such as a blister pack. Such packaging of the components separately can also, in certain instances, permit long-term storage without losing activity of the components.

Kits can also include reagents in separate containers such as, for example, sterile water or saline to be added to a lyophilized active component packaged separately. For example, sealed glass ampules can contain a lyophilized component and in a separate ampule, sterile water, sterile saline or sterile each of which has been packaged under a neutral non-reacting gas, such as nitrogen. Ampules can consist of any suitable material, such as glass, organic polymers, such as polycarbonate, polystyrene, ceramic, metal or any other material typically employed to hold reagents. Other examples of suitable containers include bottles that can be fabricated from similar substances as ampules, and envelopes that can consist of foil-lined interiors, such as aluminum or an alloy. Other containers include test tubes, vials, flasks, bottles, syringes, and the like. Containers can have a sterile access port, such as a bottle having a stopper that can be pierced by a hypodermic injection needle. Other containers can have two compartments that are separated by a readily removable membrane that upon removal permits the components to mix. Removable membranes can be glass, plastic, rubber, and the like.

In certain embodiments, kits can be supplied with instructional materials. Instructions can be printed on paper or other substrate, or can be supplied as an electronic-readable medium, such as a floppy disc, mini-CD-ROM, CD-ROM, DVD-ROM, Zip disc, videotape, audio tape, and the like. Detailed instructions cannot be physically associated with the kit; instead, a user can be directed to an Internet web site specified by the manufacturer or distributor of the kit.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

The following example shows that Taq D732N is a mutant Taq DNA polymerase (“OmniTaq 2”) that has reverse transcriptase activity, increased resistance to black pepper, greater processivity, and ability to catalyze LAMP (3). The mutation also has an unwanted effect of decreasing the signal of TaqMan® assays. It is shown that this is caused by decreased 5′-exonuclease activity.

The TaqMan® assay depends on the so-called 5′-exonuclease, which is actually an endonuclease of displaced 5′-base pairs, called a flap endonuclease. In the PCR product detection method TaqMan®, the flap nuclease cleaves the probe annealed to PCR products, allowing increased fluor detection in real time. As described herein, it was surprisingly found that by adding a very small amount of wild Type Taq Polymerase, which contains a normal amount of 5′-exonuclease activity, the TaqMan® assay was more successful.

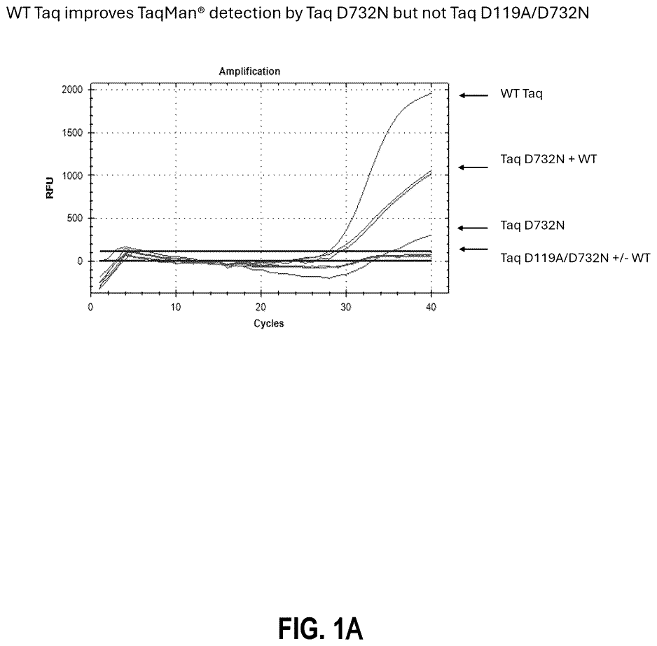

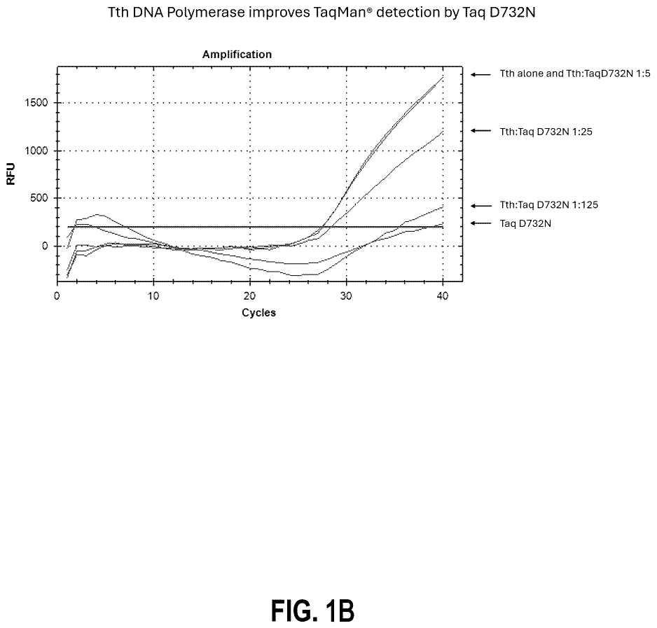

WT Taq was mixed with Taq D732N or Taq D119A/D732N in ratios of 1:25 and 1:50. Each enzyme was also tested separately. Both ratios of wild Type to D732N improved detection, which neither ratio was sufficient to allow TaqMan® detection of Taq D119A/D732N. 25 μL reactions contain 1.3M Betaine, PCR buffer, 200 nM each CCR5 240 bp primer, 200 μM dNTPs (deoxynucleotide triphosphates), 5 ng Hu DNA, 200 nM CCR5 TaqMan® probe (FAM), 0.1 μL DNA Polymerase or DNA Polymerase mixture. Cycled: 94° 2′ then (94° 40″, 59° 30″, 68° 1′)×40. All reactions yielded robust amplification as visualized on an agarose gel. FIG. 1A.

Tth DNA Polymerase was mixed with Taq D732N in ratios of 1:5, 1:25, 1:125. Each enzyme was also tested separately. Ratios of 1:5 and 1:25 significantly improved TaqMan® detection. The 25 μL reactions contained 1.3M Betaine, PCR buffer, 200 nM each CCR5 240 bp primer, 200 μM dNTPs, 5 ng Hu DNA, 200 nM CCR5 TaqMan® probe (FAM), 0.1 μL DNA Polymerase or DNA Polymerase mixture. Cycle conditions were: 94° C., 2′ then (94° 40″, 59° 30″, 68° 1′)×40. All reactions yielded robust amplification as visualized on an agarose gel. (FIG. 1B.)

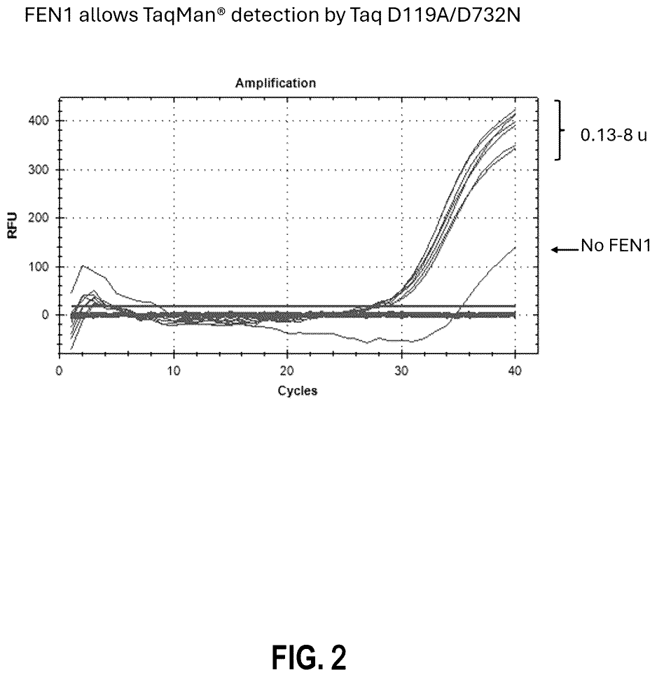

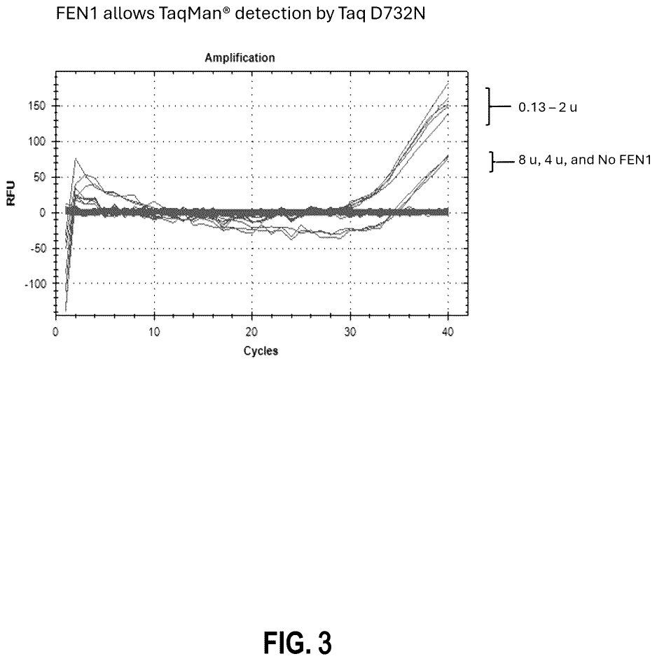

The double mutant Taq D119A/D732N has a further decrease in the 5′-exonuclease activity, beyond that noted in Taq D732N. As expected, the double mutant Taq D119A/D732N shows no signal in TaqMan® assays. The addition of wild Type Taq was not sufficient for D119A/D732N performance in the TaqMan® assay (See FIGS. 1A), but the addition of FEN1 (New England Biolabs, Inc., catalog number M0645S) enabled detection with Taq D119A/D732N in the TaqMan® assay. The 25 μL reactions contained 1.3M Betaine, PCR buffer, 200 nM each CCR5 240 bp primer, 200 μM dNTPs, 5 ng Hu DNA, 200 nM CCR5 TaqMan® probe (FAM), 0.1 μL Taq D119A/D732N or D732N, and FEN1 as indicated. Cycle conditions were: 94° C., 2′ then (94° 40″, 59° 30″, 68° 1′)×40. (See FIG. 2). In Taq D732N TaqMan® assay experiments, FEN1 (New England Biolabs, Inc., catalog number M0645S) was included from 0 to 8 units (u) per reaction. 0.13-2 u of FEN1 increased TaqMan® assay detection for Taq D732N (OmniTaq 2). 4 u and 8 u did not help. (See FIG. 3). 25 μL reactions contain 1.3 M Betaine, PCR buffer, 200 nM each CCR5 240 bp primer, 200 μM dNTPs, 5 ng Hu DNA, 200 nM CCR5 TaqMan® probe (FAM), 0.1 μL Taq D732N Polymerase. Cycled conditions were: 94° C., 2′ then (94° 40″, 59° 30″, 68° 1′)×40.and 3.) The FEN1 enzyme may have better access to the active site.

### Example 2

It was also shown that FEN1 can enable Klentaq® DNA polymerase and mutants of Klentaq® to perform in TaqMan® assays.

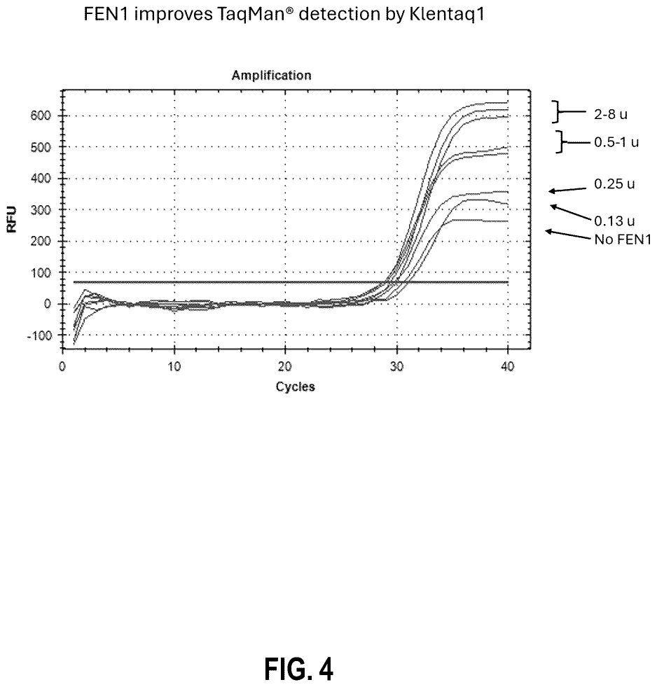

All amounts of FEN1 improved TaqMan® detection for Klentaq® with higher amounts showing the greatest improvement. The 25 μL reactions contained 1.3M Betaine, PCR buffer, 200 nM each CCR5 240 bp primer, 200 μM dNTPs, 5 ng Human DNA, 200 nM CCR5 TaqMan® probe (FAM), 0.1 μL Klentaq®1, and FEN1 (New England Biolabs, Inc., catalog number M0645S) as indicated. Cycle conditions were: 94° C., 2′ then (94° 40″, 59° 30″, 68° 1′)×40. (FIG. 4)

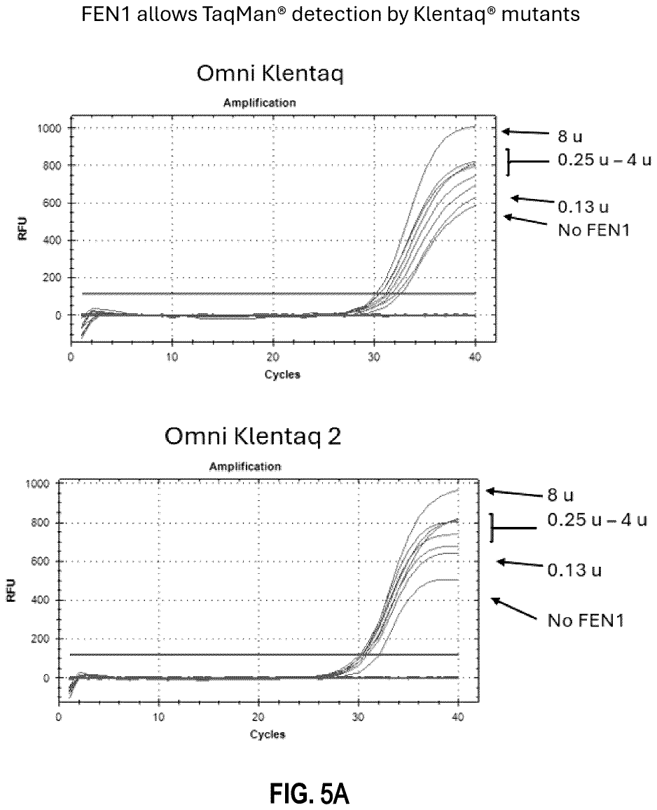

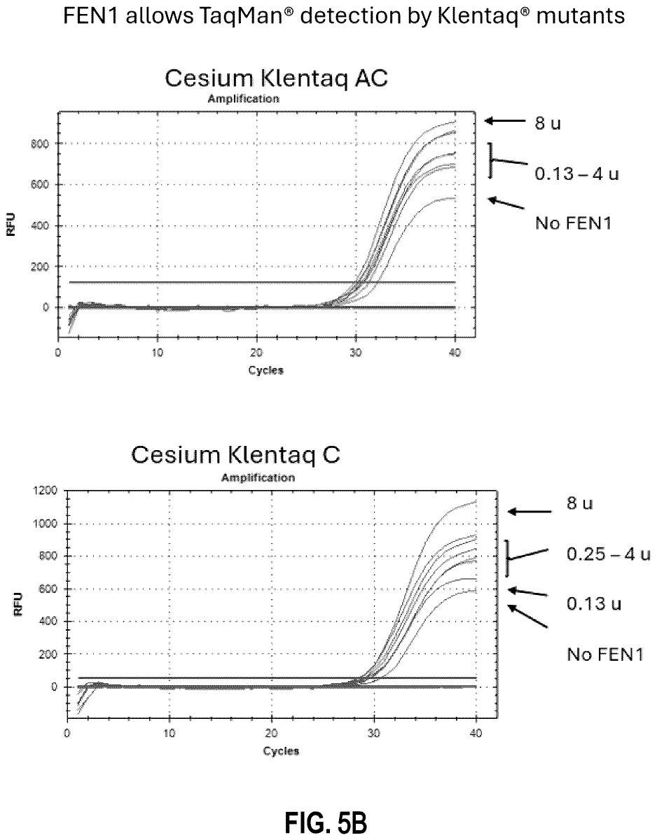

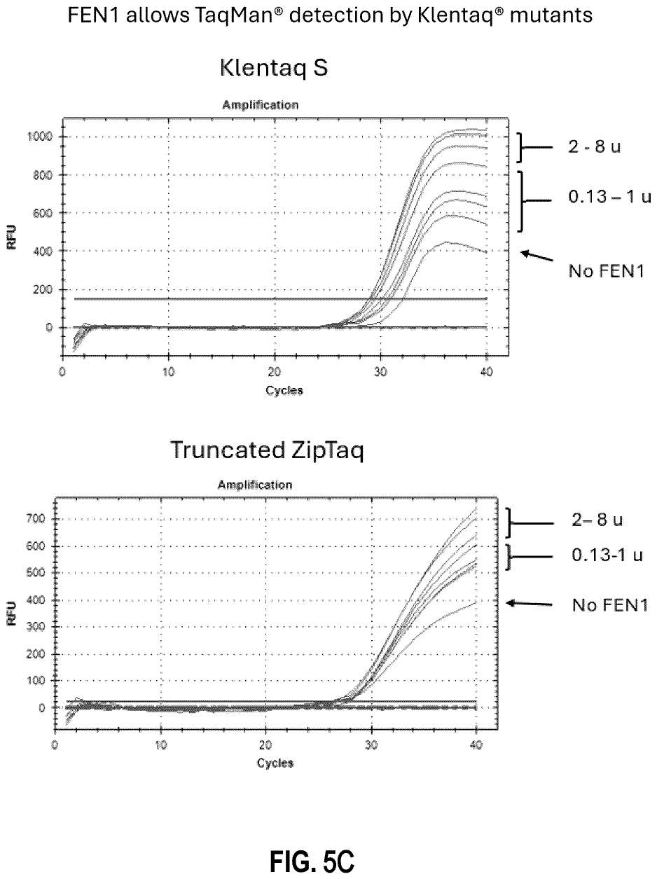

FIGS. 5A, B, and C are amplification curves showing that FEN1 allows TaqMan® detection by various Klentaq® mutants.

FEN1 (New England Biolabs, Inc., catalog number M0645S) was included from 0 to 8 u per reaction. All amounts improved detection for Omni Klentaq® and Omni Klentaq®2 with 8 u showing the most improvement for both. The 25 μL reactions contained 1.3M Betaine, PCR buffer, 200 nM each CCR5 240 bp primer, 200 μM dNTPs, 5 ng Human DNA, 200 nM CCR5 TaqMan® probe (FAM), 0.04 μL Polymerase. conditions were: 94° C., 2′ then (94° 40″, 59° 30″, 68° 1′)×40. (FIG. 5A)

FEN1 was included from 0 to 8 u per reaction with Cesium Klentaq® AC and Cesium Klentaq® C. All amounts improved TaqMan® detection for Cesium Klentaq® AC and Cesium Klentaq® C, with higher amounts showing the greatest improvement. The 25 μL reactions contained 1.3M Betaine, PCR buffer, 200 nM each CCR5 240 bp primer, 200 μM dNTPs, 5 ng Human DNA, 200 nM CCR5 TaqMan® probe (FAM), and 0.04 μL Polymerase. Cycling conditions were: 94° C., 2′ then (94° C., 40″, 59° 30″, 68° 1′)×40. (FIG. 5B).

FEN1 was included from 0 to 8 u per reaction Klentaq® S and a truncated version of ZipTaq (Klentaq® version of ZipTaq). All amounts improved TaqMan® detection for Klentaq® S and a truncated version of ZipTaq (Klentaq® version of ZipTaq) with higher amounts being most effective. 25 μL reactions contain 1.3M Betaine, PCR buffer, 200 nM each CCR5 240 bp primer, 200 μM dNTPs, 5 ng Human DNA, 200 nM CCR5 TaqMan® probe (FAM), 0.04 μL Polymerase. Cycle conditions were: 94° C., 2′ then (94° C., 40″, 59° 30″, 68° 1′)×40. (FIG. 5c).

Klentaq® is a truncated version of Taq Polymerase, which lacks the N-terminal 5′-exonuclease domain. Klentaq® is superior to wild Type Taq with respect to its thermostability and fidelity (1) as well as some inhibition resistance. Mutants of Klentaq® can confer additional features such as inhibition resistance (2) and hot-start performance (4). However, the complete lack of 5′-exonuclease activity means Klentaq®, and its mutants, are unable to perform in TaqMan® assays. The addition of FEN1 opens up the possibility of using these superior enzymes which include such additional features for TaqMan® assays.

### Example 3

Additionally, FEN1 can improve mutant DNA polymerases' performance in TaqMan® assays, even when the polymerase has intact 5′-exonuclease activity.

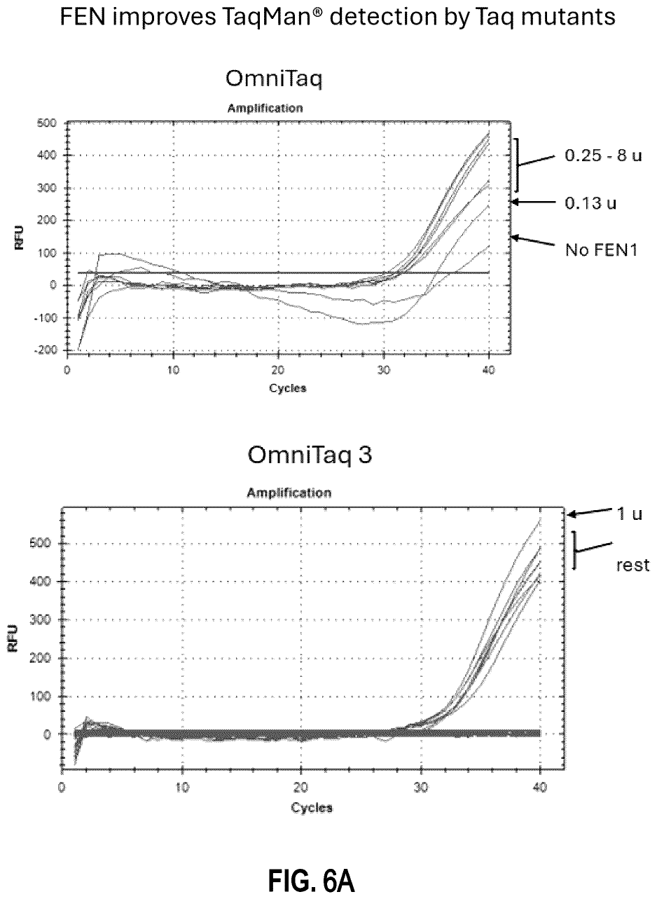

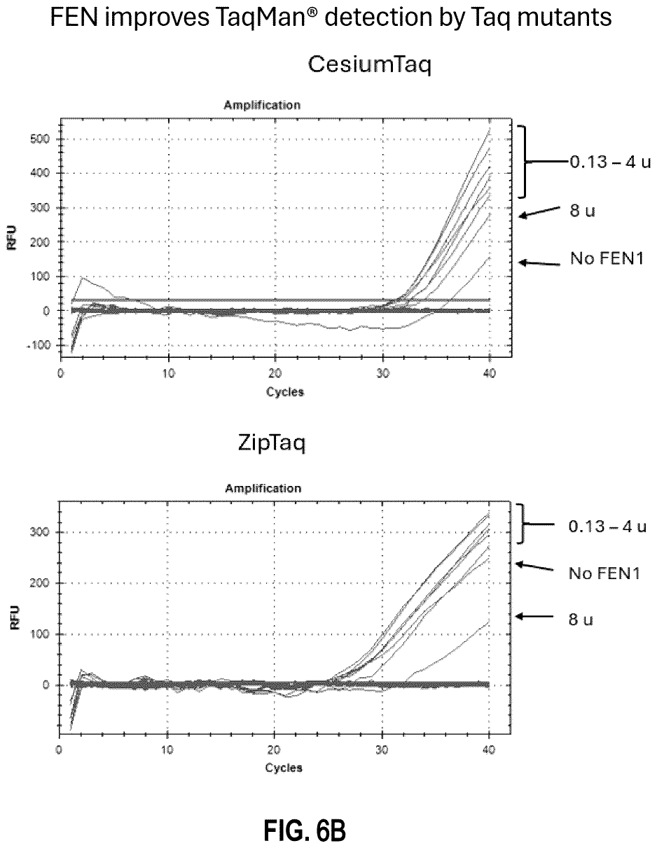

FEN1 was included from 0 to 8 u per reaction with OmniTaq and OmniTaq 3. All amounts improved TaqMan® detection for OmniTaq with 0.13 u being the least effective. For OmniTaq 3, modest improvement was noted for 0.13-4 u. 8 u was not effective. 25 μL reactions contained 1.3M Betaine, PCR buffer, 200 nM each CCR5 240 bp primer, 200 μM dNTPs, 5 ng Hu DNA, 200 nM CCR5 TaqMan® probe (FAM), 0.04 μL Polymerase. Cycled: 94° 2′ then (94° 40″, 59° 30″, 68° 1′)×40. (See FIG. 6A.)

FEN1 was included from 0 to 8 u per reaction with Cesium Taq and ZipTaq. All amounts of FEN1 boosted the TaqMan® signal for CesiumTaq, with 8 u being least effective. FEN1 provided only modest improvement in detection for ZipTaq, except for 8 u which decreased detection. The 25 μL reactions also contained 1.3 M Betaine, PCR buffer, 200 nM each CCR5 240 bp primer, 200 μM dNTPs, 5 ng Hu DNA, 200 nM CCR5 TaqMan® probe (FAM), 0.1 μL Polymerase. Cycle conditions were: 94° C., 2′ then (94° 40″, 59° 30″, 68° 1′)×40. (FIG. 6B).

Mutants of Taq Polymerase, which include the N-terminal 5′-exonuclease domain, have been shown to have improvements over wild Type Taq in inhibition resistance (2), speed (3), reverse transcription (3), strand displacement (3), and hot-start performance (4). Mutants of Taq Polymerase, which include the N-terminal 5′-exonuclease domain, perform well in TaqMan® assays. It is shown that the addition of FEN1 boosts the TaqMan® signal.

### Example 4

Furthermore, it is shown that FEN1 improves TaqMan® detection with reactions including Klentaq® enzyme (SEQ ID NO: 2) and either: (i) a DNA hot-start aptamer (GCC GGCCAATGTACAGTATTGGCCGGCTTTTGGCGGAGCGATCATCTCAGAGCATTCT TAGCGTTTTGTTCT TGT GTATGA/3InvdT/; (SEQ ID NO: 17)); or (ii) Deep Vent® DNA polymerase (New England Biolabs, Beverly, MA, USA.

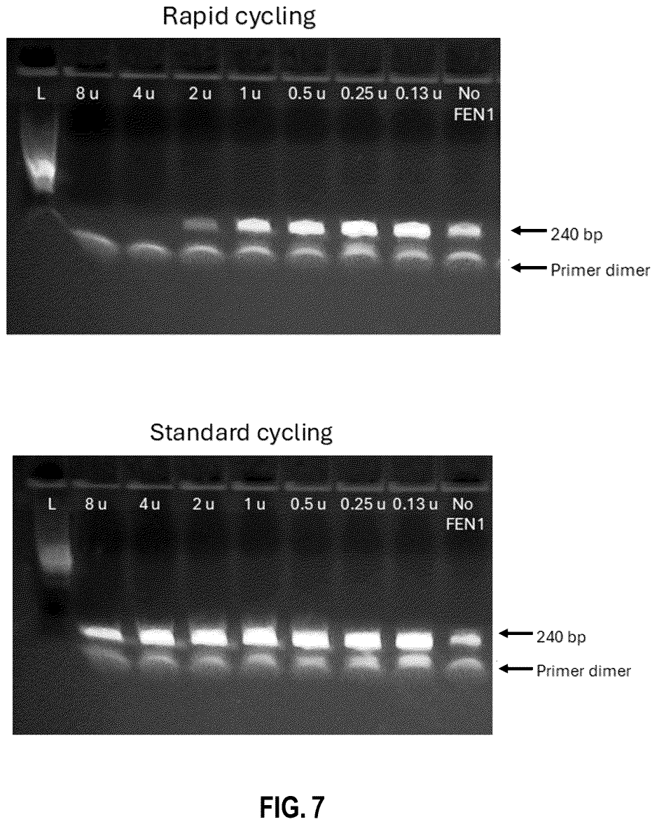

FEN1 was included from 0-8 u per reaction with Cesium Taq and ZipTaq. FEN1 not only improved TaqMan® detection (standard cycling reactions correspond to the curves shown in FIG. 6B) but also improved the yield of product for these reactions. 25 μL reactions contained 1.3M Betaine, PCR buffer, 200 nM each CCR5 240 bp primer, 200 μM dNTPs, 5 ng Hu DNA, 200 nM CCR5 TaqMan® probe (FAM), 0.05 μL Polymerase. Rapid cycling conditions were: 94° 2′ then (94° 10″, 59° 10″, 68° 10″)×40. Standard cycling conditions were: 94° 2′ then (94° 40″, 59° 30″, 68° 30″)×40. (See FIG. 7).

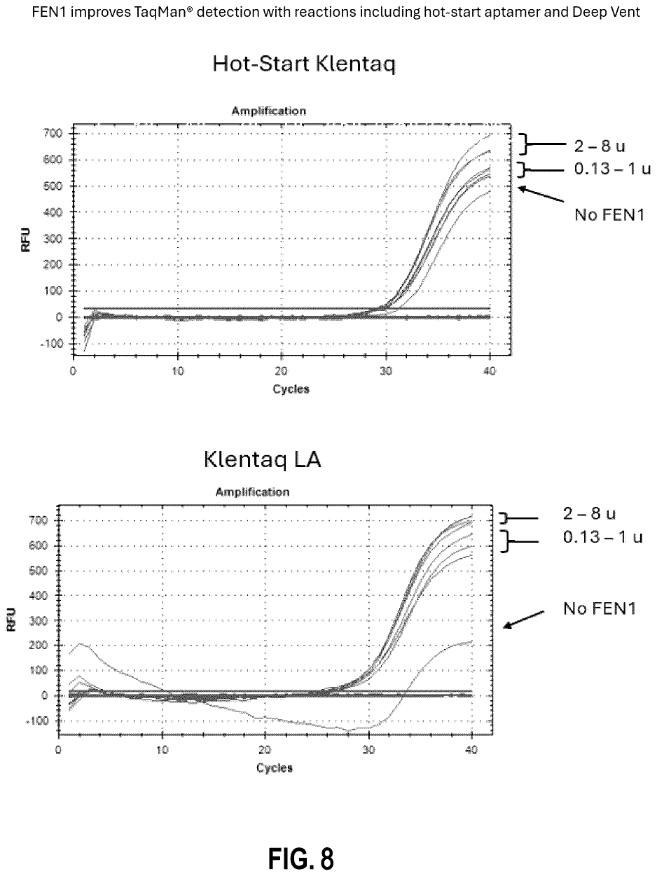

FIG. 8 show amplification curves showing FEN1 improves TaqMan® detection with reactions including hot-start aptamer and Deep Vent® DNA polymerase (New England Biolabs, Beverly, MA, USA). FEN1 was included from 0 to 8 u per reaction. All amounts of FEN1 boosted the TaqMan® signal for Hot-Start Klentaq® (Klentaq® plus DNA hot-start aptamer) and Klentaq® LA (Klentaq® plus DeepVent® DNA polymerase in a 49:1 ratio) with higher amounts of FEN1 being most effective. 25 μL reactions also contained 1.3 M Betaine, PCR buffer, 200 nM each CCR5 240 bp primer, 200 μM dNTPs, 5 ng Hu DNA, 200 nM CCR5 TaqMan® probe (FAM), 0.1 μL Polymerase. Cycled: 94° 2′ then (94° 40″, 59° 30″, 68° 1′)×40. (See FIG. 8).

As described herein, detection of PCR products in TaqMan® assays using Taq DNA Polymerases with reduced or absent 5′-exonuclease activity is improved by addition of Thermostable Flap Endonuclease 1 (FEN1) (New England Biolabs, Inc., catalog number M0645S) to the TaqMan® assays and/or addition of WT Taq DNA Polymerase or other DNA Polymerases with 5′-exonuclease activity. In various embodiments, the amount of FEN1 in the TaqMan® assays can be between 0.13 and 8 u (units) per 25 μL PCR reaction. One unit of FEN1 activity is defined as the amount of enzyme required to cleave 10 μmol of 5′ flap containing oligonucleotide substrate in a total reaction volume of 10 μl for 10 min at 65° C. In various embodiments, the amount of WT Taq DNA Polymerase in the TaqMan® assays is between 1/25 to 1/50 of the amount of mutant Taq DNA polymerase.

Detection of PCR products in TaqMan® assays using Taq DNA Polymerases with typical (e.g. wild type) 5′-exonuclease activity is improved by addition of Thermostable Flap Endonuclease 1 (FEN1) to the TaqMan® assays. In some embodiments, Taq can be a mutant such as OmniTaq (Taq E626K/1707L/E708N), OmniTaq3 (Taq E818V), or ZipTaq (Taq A391T/D732N/E742R/A743R/P752S/E818V). In various embodiments, the amount of FEN1 in the TaqMan® assays can be between 0.13 and 8 u per 25 μL PCR reaction.

Yield of PCR products in TaqMan® assays using Taq DNA Polymerases is increased by addition of Thermostable Flap Endonuclease 1 (FEN1) to the TaqMan® assays. For example, increased yield can be improved if the Taq DNA polymerase is ZipTaq (Taq A391T/D732N/E742R/A743R/P752S/E818V). In various embodiments, the amount of FEN1 in the TaqMan® assays can be between 0.13 and 8 u per 25 μL PCR reaction.

Detection of PCR products in TaqMan® assays using Taq DNA Polymerases with reduced or absent 5′-exonuclease activity is improved by addition of Thermostable Flap Endonuclease 1 (FEN1) to the TaqMan® assays when: (i) the Taq DNA Polymerase has an N-terminal deletion of Taq (Klentaq®); (ii) the Taq DNA Polymerase is a mutant of Klentaq®; and/or (iii) the Taq DNA Polymerase is a mutant with reduced or eliminated 5′-exonuclease activity.

Detection of PCR products in TaqMan® assays using Taq DNA Polymerases with reduced or absent 5′-exonuclease activity is improved by addition of Thermostable Flap Endonuclease 1 (FEN1) to the TaqMan® assays when the Taq DNA Polymerase is a mutant with reduced or eliminated 5′-exonuclease activity, such as when the mutant is OmniTaq 2 (Taq D732N) or Taq D119A/D732N.

Detection of PCR products in TaqMan® assays using Taq DNA Polymerases with reduced or absent 5′-exonuclease activity is improved by addition of Thermostable Flap Endonuclease 1 (FEN1) to the TaqMan® assays when the Taq DNA Polymerase is a mutant of Klentaq®, such as when Taq is Omni Klentaq (Klentaq® E626k/1707L/E708K), Omni Klentaq2 (Klentaq® K738R), Cesium Klentaq AC (Klentaq® E626k/1707L), Cesium Klentaq C (Klentaq® 1707L), Klentaq®-S(Klentaq® F667Y), or a truncated version of ZipTaq (Klentaq® A391T/D732N/E742R/A743R/P752S/E818V).

What is claimed is:

1. A method for improving detection of Polymerase Chain Reaction (PCR) products, comprising:
adding a mutant Thermus aquaticus (Taq) DNA polymerase to a PCR mixture comprising a polynucleotide template, primers, and deoxynucleotide triphosphates which can amplify the polynucleotide template, wherein the mutant Taq DNA polymerase comprises a mutation that reduces or eliminates 5′-exonuclease activity of Taq; and
adding a 5′-exonuclease enzyme to the PCR mixture.

2. The method of claim 1, wherein the detection of the PCR products is in a probe degradation assay.

3. The method of claim 2, wherein the probe degradation assay uses a sequence-specific DNA probe including a fluorescent reporter at one end of the probe and a quencher of fluorescence at the opposite end of the probe.

4. The method of claim 1, wherein the 5′-exonuclease enzyme is a thermostable flap endonuclease 1 (FEN1).

5. The method of claim 4, wherein the amount of FEN1 in the PCR mixture is between 0.13 and 8 units of FEN1 per 25 μL PCR reaction.

6. The method of claim 1, wherein the mutant Taq DNA Polymerase has an N-terminal deletion of the wild-type Taq DNA polymerase and comprises an amino acid sequence having at least 95% sequence identity to SEQ ID NO: 2, but does not comprise the amino acid sequence of SEQ ID NO: 2.

7. The method of claim 6, wherein the mutant comprises amino acid substitutions corresponding to: (i) E626K, I707L, and E708K; (ii) K738R; (iii) E626K and I707L; (iv) I707L; (v) F667Y; or (vi) A391T, D732N, E742R, A743R, P752S, and E818V; and wherein the amino substitutions are in reference to SEQ ID NO: 1.

8. The method of claim 1, wherein the mutant Taq DNA Polymerase comprises an amino acid sequence having at least 95% sequence identity to SEQ ID NO: 1 and comprises amino acid substitutions in SEQ ID NO: 1 corresponding to (i) E818V; (ii) A391T, D732N, E742R, A743R, P752S, and E818V; (iii) D732N; or (iv) D119A and D732N.

9. The method of claim 1, wherein the 5′-exonuclease enzyme is a wild-type DNA Polymerase.

10. The method of claim 9, wherein the amount of WT Taq DNA Polymerase in the PCR reaction mixture is between 1/25 to 1/50 of the amount of mutant Taq DNA polymerase in the PCR reaction mixture.

11. A method for increasing yield of PCR products in a PCR reaction, comprising:
adding a mutant Taq DNA polymerase to a PCR reaction mixture comprising a polynucleotide template, primers which can amplify the polynucleotide template, and deoxynucleotide triphosphates; and
adding a 5′-exonuclease enzyme to the PCR reaction mixture.

12. The method of claim 11, wherein the mutant Taq DNA polymerase comprises an amino acid sequence having at least 95% sequence identity to SEQ ID NO: 1 and comprises amino acid substitutions corresponding to A391T,D732N, E742R, A743R, P752S, and E818V in SEQ ID NO: 1.

13. The method of claim 11, wherein the 5′-exonuclease enzyme is a thermostable flap endonuclease 1 (FEN1).

14. The method of claim 13, wherein the amount of FEN1 in the TaqMan® assays is between 0.13 and 8 units of FEN1 per 25 μL PCR reaction.

15. The method of claim 13, wherein the mutant Taq DNA Polymerase has an N-terminal deletion of the wild-type Taq DNA polymerase and comprises an amino acid sequence having at least 95% sequence identity to SEQ ID NO: 2, but does not comprise the amino acid sequence of SEQ ID NO: 2.

16. A PCR mixture comprising:
(i) a mutant Taq DNA polymerase, wherein the mutant Taq DNA polymerase comprises a mutation that reduces or eliminates 5′-exonuclease activity of Taq;
(ii) a 5′-exonuclease enzyme; and
(iii) a polynucleotide template, primers which can amplify the polynucleotide template, and deoxynucleotide triphosphates.

17. The PCR mixture of claim 16, wherein mixture further comprises a sequence-specific DNA probe including a fluorescent reporter at one end of the probe and a quencher of fluorescence at the opposite end of the probe.

18. The PCR mixture of claim 15, wherein the 5′-exonuclease enzyme is a thermostable flap endonuclease 1 (FEN1).

19. The PCR mixture of claim 18, wherein the amount of FEN1 in the PCR mixture is between 0.13 and 8 units of FEN1 per 25 μL PCR reaction.

20. The PCR mixture of claim 15, wherein the mutant Taq DNA Polymerase has an N-terminal deletion of the wild-type Taq DNA polymerase and comprises an amino acid sequence having at least 95% sequence identity to SEQ ID NO: 2, but does not comprise the amino acid sequence of SEQ ID NO: 2.
